(19) United States
(12) Plant Patent Application Publication
(10) Pub. No.: US 2002/0095706 P1
(43) Pub. Date: Jul. 18, 2002

(54) Begonia named begotis 99-2

(76) Inventor: Monica Sanders, Grootebroek (NL)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 09/764,793

(22) Filed: Jan. 17, 2001

(51) Int. Cl. A01H 5/00

(52) U.S. Cl. PLT/343

(57) ABSTRACT

A new Begonia plant particularly distinguished by its large, silvery white flower with intensive rose backside in outdoor plantings, giving a bicolor effect, bronze dark red leaves, early flowering, an erect habit and easily self branching.

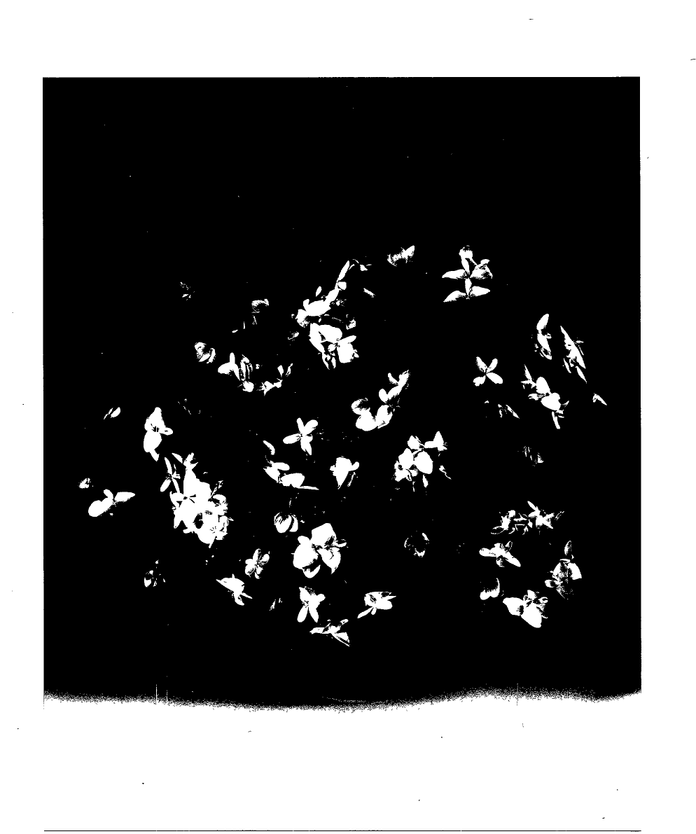

### BACKGROUND OF THE NEW PLANT

The present invention comprises a new distinct cultivar of Begonia, botanically known as a cross between B. obliqua and B. semperflorens. The new cultivar is propagated from cuttings resulting from the cross of P5106 and P 629. P5106 is a white flowering, green leaved Begonia obliqua and is not commercially available and is not known by any synonyms. P 629 is a B. semperflorens and a plant from the commercial variety Milo red. Neither P5106 or P 629 has been patented. As a result of this cross the present cultivar was created in 1996 in Enkhuizen, Netherlands and has been repeatedly asexually reproduced by cuttings in Enkhuizen, Netherlands over a four-year period. It has been found to retain its distinctive characteristics through successive propagations, and this novelty appears to be firmly fixed. This new Begonia plant is an annual in most climatical zones in the US.

### DESCRIPTION OF THE DRAWING

This new Begonia plant is illustrated by the accompanying photographic drawing which shows blooms, buds and foliage of the plant in full color, the color shown being as true as can be reasonably obtained by conventional photographic procedures.

### DESCRIPTION OF THE NEW CULTIVAR

The following detailed descriptions set forth the distinctive characteristics of this new Begonia. The data that defines these characteristics were collected from asexual reproductions carried out in Enkhuizen, Netherlands. The plant history was taken on 1 year old plants, blossomed under natural light in a greenhouse. Color readings were taken in the office under natural daylight. Color references are primarily to The RHS Color Chart of the Royal Horticultural Society of London.

The Plant:

Classification.—Botanical: Begonia obliqua×semperflorens

Parentage.—Female parent: A seedling named P5106 is one of our seedlings from our full sib-generation of plants bred in 1995 —Pollen parent: A seedling named P 629 is one of our seedlings from Milo red.

Growth habit.—erect, later spreading.

Plant height.—up to 80 cm.

Spreading area of plant.—up to 1 m.

Growth rate.—vigorous, easily self branching.

Strength.—Good.

Branching character.—Good branching.

Blooming period.—continuously, 4 weeks after potting the cuttings.

The stem:

Diameter.—3-6 mm.

Shape.—Round.

Anthocyan pigmentation.—present.

Length of internode.—20-80 mm, depending on the light where the plant is propagated.

Pubescence.—no pubescence on the stem.

The Foliage:

Phyllotaxis.—alternate.

Shape of blade.—ovate, uneven based.

Texture.—smooth.

Edge.—dentate.

Length.—60-85 mm.

Width.—25-37 m.

Color.—Upper side: deep brown 200A —Lower side: brown red 187A.

Pubescence.—some pubescence is present.

Length of leaf stem 8-22 mm.

The Bud:

Peduncle length.—58-63 mm, depending on season.

Bud size of male flower.—Width: 12-17 mm —Length 18-22 mm.

Shape.—ovate.

Color.—nearly white 150D.

Sepals.—no sepals.

The Flower:

Direction.—Hanging.

Outward curvation of petal.—flat.

Male flower.—form: cross shaped —length: 37-53 mm —width: 34-45 mm.

Female flower.—form: star shaped —diameter: 24-38 mm.

Borne.—axillary.

Cluster.—cyme.

Color of male flower.—front side: white —back side: rose 63B.

Color of female flower.—white.

Overlapping of petals.—Separate.

No. of petals.—male 4 and female 5.

Shape of the petals of the male flower.—oval, 2 big and 2 smaller.

Size of the big petal.—length: 18-29 mm —Width: 14 -18 mm.

Size of the small petal.—length: 18-23 mm —Width: 8-11 mm.

Size of the petal of the female.—length: 16-18 mm —Width: 7-9 mm.

No. of flowers per cyme.—4-5 male flowers and 2-3 female flowers.

Reproductive organs.—male flowers have stamens, female flowers have a three-lobbed stigma and receptacle under flower.

Roots:

Type of roots.—Fibrous.

Physiological and ecological characteristics.

Good tolerance to heat and cold. Strong resistance to pests and diseases,

What is claimed is:

1. A new and distinct variety of begonia plant, substantially as herein illustrated and described, characterized particularly as to novelty by large, silvery white flowers, which have a bicolor effect in outdoor plantings, on bronze dark red foliage, flowers appear earlier on the plant, and a growing habit that is first erect but later spreading and easily self branching:
